(19) United States
(12) Patent Application Publication
(10) Pub. No.: US 2003/0143524 A1
(43) Pub. Date: Jul. 31, 2003

(54) Method and system for determining the amount, distribution and conformation of genetic material in a cell

(76) Inventor: Boaz Lerner, Mezkeret Batia (IL)

Correspondence Address: BROWDY AND NEIMARK, P.L.L.C., 624 NINTH STREET, NW, SUITE 300, WASHINGTON, DC, 20001-5303, US

(21) Appl. No.: 10/059,375

(22) Filed: Jan. 31, 2002

(51) Int. Cl. C12Q 1/70; C12Q 1/68; G06F 19/00; G01N 33/48; G01N 33/50

(52) U.S. Cl. 435/5; 435/6; 702/20

(57) ABSTRACT

A method and system for genetic analysis of cells or nuclei. For each of one or more DNA sequences in the cells or nuclei, the DNA sequence is labeled with a probe producing a detectable optical signal. A two-dimensional image of the cell or nucleus is obtained that is focused in each of a plurality of parallel planes passing through the cell or nucleus. The number of optical signals from the DNA sequence in each image having one or more predetermined characteristics is determined, and the total number of optical signals in the images having the one or more predetermined characteristics is then determined.

### FIELD OF THE INVENTION

This invention relates to optical methods for genetic analysis

### PRIOR ART

The following publications are considered relevant for proper

### U.S. PATENTS

U.S. Pat. No. 6,221,607 Tsipouras et al.

U.S. Pat. No. 5,523,207 Kamentsky & Kamentsky

### OTHER PUBLICATIONS

Amiel, A., Litmanovitch, T., Lishner, M., Mor, A., Gaber, E., Fejgin, M. D., Avivi, L. (1998a). Genes, Chromosomes & Cancer, 22, 225-231.

Amiel, A., Kolodizner, T., Fishman, A., Gaber, E., Klein, Z., Beyth, Y., Fejgin, M. D. (1998b). Cancer, 83, 1966-1971.

Amiel, A., Korenstein, A., Gaber, E., Avivi, L. (1999). Eur J Hum Genet, 7, 223-230.

Amiel, A., Kitay-Cohen, Y., Fejgin, M. D., Lishner, M. (2000). Exp Hemal, 28, 156-160.

Amiel, A., Elis, A., Blumenthal, D., Gaber, E., Fejgin, M. D., Dubinsky, R., Lishner, M. (2001). Cancer Genet Cytogenet, 125, 156-160.

Bar-AM, I., Mor, O., Yeger, H., Shiloh, Y., Avivi, L. (1992). Genes, Chromosomes & Cancer, 4, 314-320.Bishop, C. M. (1995).“Neural Networks for Pattern Recognition”, Clarendon Press, Oxford.

Castleman, K. R. and White, B. S. (1995). Bioimaging, 3, 88-93.

Dotan, A. Z., Dotan, A., Litmanovitch, T., Ravia, Y., Oniashvili, N., Leibovitch, I., Ramon, J., and Avivi L. (2000). Genes, Chromosomes & Cancer, 27, 270-277.

Finkelstein, S., Mukamel, E., Yavetz., H., Paz, G., Avivi, L. (1998). Hum Genet, 102, 129-137.

Klinger, K., Landes, G., Shook, D., Harvey, R., Lopez, L., Locke, P., Lerner, T., Osathanondh, R, Leverone, B., Houseal, T., Pavelka, K., Dackowski, W. (1996). Am. J. Hum. Genet, 51, 99-103.

Knoll, J. H. M., Cheng, S. D., Lalande, M. (1994). Nat Genet, 6, 41-46.

Lengauer, C., Kinzler, K. W., Vogelstein, B. (1997). Nature, 386, 623-627.

Lerner, B., Clocksin, W. F., Dhanjal, S., Hult'en, M. A., and Bishop, C. M. (2001). Cytometry, 43, 87-93.

Netten, H., Young, I. T., van Vliet, L. J., Tanke, H. J., Vroljik, H., and Sloos, W. C. R. (1997). Cytometry, 28, 1-10.

Selig, S., Okumura, K., Ward, D. C., and Cedar, H. (1992). EMBO J, 11, 1217-1225.

Simon, I., Tenzen, T. Reubinoff, E., Hillman, D., McCarrey, J. R. and Cedar, H. (1999). Nature, 401, 929-932.

Tanke, H. J., Florijn, R. J., Wiegant, J. Raap, A. K., and Vrolijk, J. (1995). Histochemical Journal, 27, 4-14.

Tkachuk, D. C., Westbrook, C. A., Andreeff, M., Donlon., T. A., Cleary, M. L., Suryanarayan, K., Homge, M., Redner, A., Gray, J., Pinkel, D. (1990). Science, 250, 859-862.

Vysis catalog, 1998, p. 24

Yeshaya, J., Shalgi, R., Shohat, M., and Avivi, L. (1998). Hum Genet, 102, 6-14.

Yeshaya, J., Shalgi, R., Shohat, M., and Avivi, L. (1999). Hum Genet, 105, 86-97.

### URLs

Applied Imaging—http://www.cytovision.com/

Ikonisys—http://www.ikonisys.com/

Zeiss—http:///www.zeiss.com/

MetaSystems—http://www.metasystems.de/

### BACKGROUND OF THE INVENTION

Fluorescence in-situ hybridisation (FISH) is a method used in the analysis of genetic material in a specimen. The specimen may consist of, for example, one or more metaphase or interphase cells or nuclei spread on a slide or fixed in suspension so as to preserving their three-dimensional (3D) structure. In this method, a fluorescent label, or fluorophore, is used that specifically labels a particular DNA sequence, i.e., a whole chromosome, part of a chromosome or even a specific gene. A labelled DNA sequence produces an optical signal referred to herein as a “dot” or “signal” that is detectable using an optical system typically including a microscope. FISH allows the detection of specific DNA sequences. By counting the number of dots in an image of the cell, it is possible to determine the number of copies of the DNA sequence present in a cell. When followed by signal counting, and analysis of signal type distribution and conformation in a population of cells, FISH permits identification, analysis and quantification of specific numerical and structural chromosomal abnormalities, as well as single gene disorders. For example, dot counting is employed in detecting aneuploidy (an excess or deficit of a whole chromosome) as well as in revealing sub-chromosomal deletions and duplications, and chromosomal micro-deletions (Klinger et al., 1996). Aneuploidy causes numerous genetic malformations such as Dow syndrome (trisomy of chromosome 21), trisomies of chromosomes 13 and 18, and various types of sex chromosome imbalance. Moreover, sub-chromosomal deletions, duplications, and chromosomal micro-deletions are revealed in various types of mental retardation and congenital malformations. An example of a micro-deletion detectable by dot counting is DiGeorge syndrome (Vysis catalog, 1998, p. 24). Dot counting has also been useful and largely employed for the analysis of gene amplification in cancer (Bar-Am et al., 1992), for studying solid tumour biology (Lengauer et al., 1997) and for estimating semen quality, a parameter associated with male fertility (Finkelstein et al., 1998). FISH dot counting has further been expanded to enable the detection of translocations (abnormal distribution of DNA sequences on chromosomes). FISH translocation analysis, which relies on the number of signals and the spatial pattern of their distribution, is best exemplified in the analysis of specific translocations characterising blood malignancies (Tkachuk et al., 1990).

Moreover, it is known today that adequate genetic information (gene expression) is not preserved merely by the normal amount and proper distribution of the various DNA sequences within the cell but also by the timing of DNA replication (Selig, et al., 1992). During the replication stage of the cell cycle, FISH signal conformation is a marker for estimating replication timing of any given DNA sequence (Selig, et al., 1992; Simon et al., 1999). Accordingly, a conformation of a single dot-like signal (“singlet”; S) is indicative of a DNA sequence before replication, whereas a bipartite signal assuming a conformation of two closely associated dots (“doublet”; D) indicates a sequence that has completed replication (Selig, et al., 1992). During the course of replication, FISH reveals two additional non-dot like signals (R1 and R2), the first assumes a large dispersed spot and the second displays a rod-like conformation (Yeshaya et al., 1999).

Thus, following FISH, an early replicating DNA sequence is evident as a high frequency of cells in which both alleles have been replicated (DD cells), whereas a late replicating sequence is evident as a high frequency of cells showing two non-replicated alleles (SS cells). Moreover, FISH enables enables of replication timing of one allele relative to its counterpart. Accordingly, a pair of alleles replicating synchronously (bi-allelic mode) is revealed as a low frequency of cells with either one replicated and one non-replicated allele (SD cells), or two dissimilar signals. In contrast two allelic counterparts replicating asynchronously (mono-allelic mode) is revealed as a large frequency of SD cells or cells with two dissimilar signals. Several genetic diseases are associated with delayed replication of a gene (e.g., the mutated allele in fragile-X mental retardation syndrome (FMR1) (Yeshaya et al., 1998)). Similarly, loss of the mono-allelic mode of replication, is associated with congenital malformations (e.g., in the Prader-Willi/Angelman syndrome (Knoll et al., 1994) as well as with malignancies (e.g., renal cell carcinoma (Dotan et al., 2000)). In addition, a line of evidence suggests that also loss of the bi-allelic mode of replication is displayed in various types of cancer for many bi-allelically expressed genes (e.g., in leukemia and lymphoma (Amiel et al., 1998a; Amiel et al., 2000), renal cell carcinoma (Dotan. et al., 2000), and carcinoma of the cervix (Amiel et al., 1998b)). Thus, diagnosing the frequencies of SS, DD and SD cells together with analysing in details FISH signal conformation is a means for the detection of various genetic abnormalities as well as a tool for following various types of genetic disorders characterising cancer.

A large number of cells is required for FISH dot counting in order to achieve an accurate estimation of the proportion of chromosomes over a cell population, especially in applications involving a relatively low frequency of abnormal cells. Thus, dot counting is preferably automated using digital microscopy to capture images of the cells together with image analysis and pattern recognition techniques applied to the images for dot detection and counting (Castleman & White, 1995; Netten et al., 1997; Tanke, 1995).

DNA sequences are 3D structures that are distributed within 3D cells. A two-dimensional image of a cell captured by an imaging system at a specific focal plane passing through the cell can only represent DNA sequences lying in or near to this plane. Therefore, for automated dot counting, it is necessary to obtain a plurality of images in different focal planes, and then selecting an image showing a particular signal. One method to select an image, known as “automatic focus” attempts to select the sharpest or most focused image as the focal plane of the images is shifted along an axis perpendicular to the focal planes (referred to herein as the “Z-axis”) (Netten et al., 1997). However, automatic focus can fail if the mechanism focuses on a source of noise such as debris or background fluorescence, or if the field of view (FOV) is empty (Netten et al. 1997). Therefore, subsequent manual inspection for discarding such images is sometimes necessary. Moreover, the sharpest image, even if found, can only represent a specific 2D-section of the 3D-cell. Signals in other sections of the cell, above or below the selected section, do not participate in the analysis. Thus, the selected image is actually only one of a several sharpest images for the FOV. FIG. 1, for example, shows two images of a cell population obtained at different focal planes of the same FOV. As can be seen, each image contains different focused signals. A method that selects only one image utilizes only a small amount of the total available structural information. This is the main cause of the relatively high error rate of the automatic focus method (Netten et al., 1997). The computational load involved in performing automatic focus has been found to be time-consuming, i.e., between 50% and 75% of the total time needed for analysing one FOV (Netten et al., 1997).

Lerner et al., 2001 discloses use of a signal classifier configured to discriminate between in and out-of-focus signals in images taken in a FOV at different focal planes. The discrimination is performed by a neural network which classifies the signals of each image as focused or unfocused. An image that contains no unfocused signals is selected for dot counting. However, as with the automatic focus method, the signal classifier also selects only a single image and the selected image may be only one of several images not having unfocused signals.

It is also known to use image-processing operators such as deconvolution to restore an image blurred by scattered light from other focal planes. Dot counting is performed on the image created from the combination of restored images (Carl Zeiss, Germany, http://www.zeiss.com/; MetaSystems, Germany, http://www.metasystems.de/). Deconvolution is possible if the point spread function (PSF) of the system optics, which describes image degradation and unwanted light scatter, is known, or can be estimated. Since the PSF is usually unknown, estimation is required. Thus, the deconvolution is approximate so that only an approximate reconstruction of the 3D information necessary for dot counting is obtained. Furthermore, the parameters needed for the computation of the PSF are measured or extracted interactively by the system from the operator, and thus the system cannot be automated.

### SUMMARY OF THE INVENTION

The present invention provides a system and method for performing a genetic analysis of a 3D space such as a cell, nucleus, or virus. The cell or nucleus may be, for example, a metaphase or interphase cell or nucleus. Each of one or more DNA sequences in the space is labelled with a probe specific for the sequence which produces a detectable optical signal. When two or more probes are used, probes are used such that each probe produces a unique, distinguishable optical signal. The probe may be, for example, a fluorescent probe that emits light having specific spectral properties when excitated. In accordance with the invention, a two-dimensional image of the space is obtained in each of a plurality of parallel focal planes passing through the space. The images are obtained using an optical system preferably including a CCD camera mounted on a microscope. The focal planes of the images may be, for example, equally spaced or randomly selected.

For each probe used, all optical signals produced by the probe in all of the images are detected. Each detected optical signal is then classified as having or not having a predetermined characteristic. The predetermined characteristic is preferably that the signal is in focus. Classification results from each focal plane are accumulated and spatially correlated among the plurality of images permitting the detection of all signals within the space having the predetermined characteristic. The total number of signals in the space is then determined. This procedure may be repeated for a large number of spaces in a population of spaces, and the proportion of spaces having different numbers of signals may be determined.

In a preferred embodiment, classification of the signals is performed by an automated classifier. Signals are represented by a set of multi-dimensionally discriminative features and classified on the basis of these features. The classifier may be any machine learning model, such as a neural network (NN), Bayesian neural network, Bayesian network, linear model, k-nearest neighbour, or support vector machine. The classifier is trained to distinguish between the classes through the presentation of a large number of examples of signals of the different classes together with their classification as determined by an expert. Typically, several hundred training epochs are used to train the classifier. Methods for training the classifier and applying the classifier to an image are disclosed in Lerner et al., 2001. The trained classifier is then applied to discriminate between signals of routine clinical FISH images.

The invention also provides a method for the optimization of the number and type of the features upon which the classification is based. According to this aspect of the invention, the number of features used by the classifier is the number of features that minimizes the probability of classifier miscalculation. The optimal features are those maximizing the ratio of the between-class scatter to the within-class scatter.

The invention may be used detecting aneuploidy such as trisomies 13, 18 and 21 (Down's syndrome), sex chromosome imbalance, solid tumors, haematological malignancies and poor semen quality. The invention may also be used in detecting sub-chromosomal deletions and duplications, gene amplification (present for example in cancer) and micro-deletions such as are present, for example, in the DiGeorge syndrome. The invention may also be used in the detraction of loss of the mono-allelic imprinted mode of gene replication (e.g., in the Prader-Willi/Angelman syndrome), loss of the Mendelian bi-allelic mode of gene replication (e.g., in leukemia and lymphoma), renal cell carcinoma, carcinoma of the cervix, and tri-nucleotide amplification (e.g., in fragile-X mental retardation syndrome (FMR1)).

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows two FISH images of the same FOV different focal planes, showing different focused and unfocused signals in each of the images;

FIG. 2 shows a system for analyzing the genetic content of cells in accordance with one embodiment of the invention;

FIG. 3 shows a flow chart of a method for analyzing the genetic content of cells in accordance with one embodiment of the invention;

FIG. 4 shows diagnosis of trisomy 13 performed in accordance with the invention; and

FIG. 5 shows diagnosis of trisomy 21 performed in accordance with the invention.

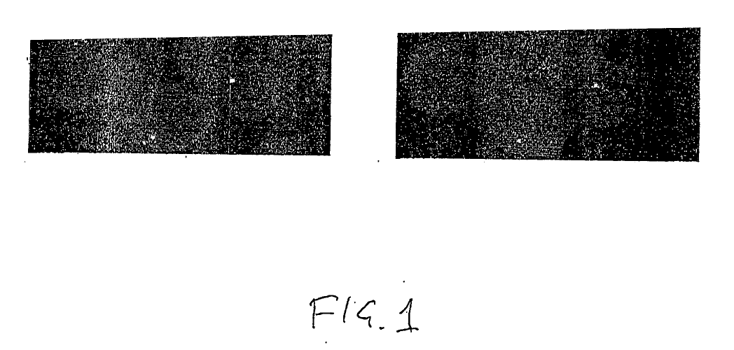

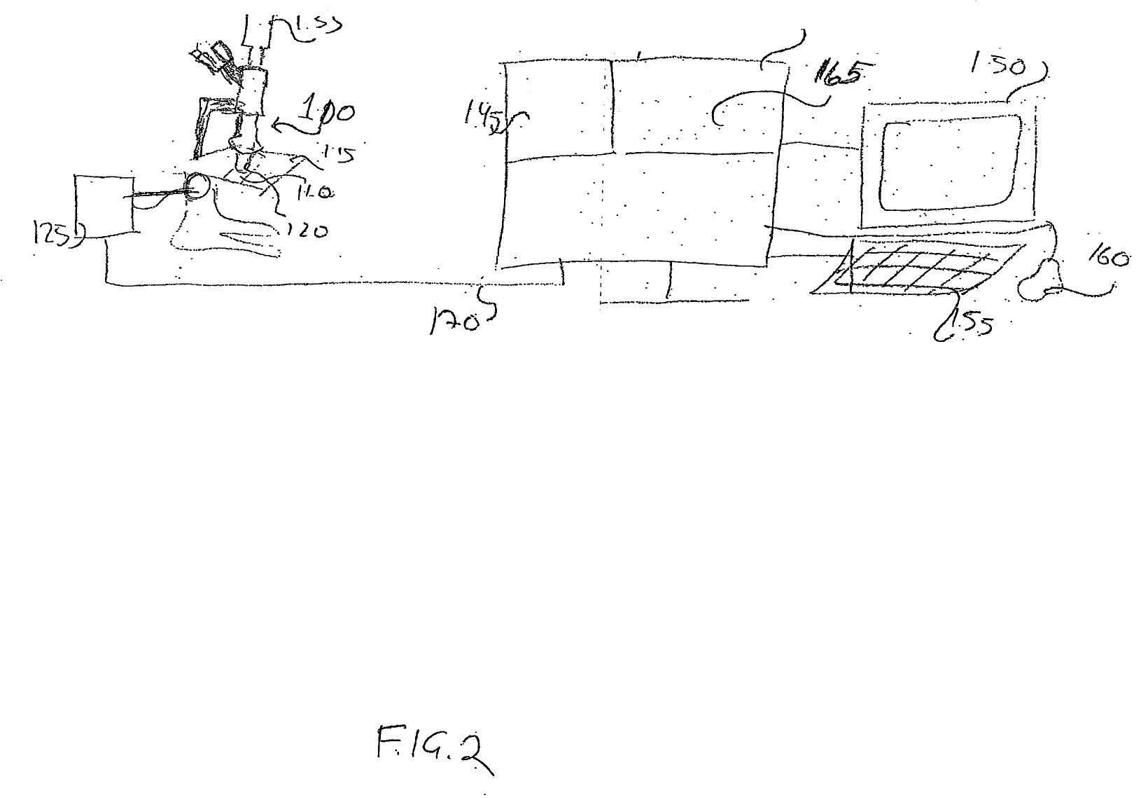

### DETAILED DESCRIPTION OF A PREFERRED EMBODIMENT OF THE INVENTION

Referring first to FIG. 2, a system is shown in accordance with one embodiment of the invention. A microscope 100 is used to from an image of a specimen 110. The specimen 110 consists of one or more three-dimensional spaces such as cells or nuclei. Each space in the specimen 110 has been labeled with one or more probes, each of which labels a specific DNA sequence. The focal plane of an image produced by the microscope 100 is selected by positioning a stage 115 of the microscope 100 relative to an objective lens 120 mounted on the microscope 100, so that the desired focal plane of the image is coincident with the focal plane of the objective lens 120. Position of the stage 115 is preferably performed by an electronic stage control 125 under the control of a CPU 130 to be described in detail below. An image of the specimen 110 produced by the microscope 100 is captured by a CCD camera 135 mounted on the microscope 100. A captured image is conveyed over a signal line 140 to the CPU 130 and may be stored in a memory 145 in the CPU 130. An image captured by the CCD camera 135 may be displayed on a display device, such as a CRT screen 150. Information may be input to the CPU 130 using any known input device such as a keyboard 155 or a computer mouse 160. The inputted information may specify, for example, the number and locations of the planes at which images are to be obtained, the spectral characteristics of the signals to be detected, or the tests to be applied to the detected signals, and the features of the signal upon which the classification is to be based.

The CPU 130 includes a processor 165 configured to activate the electronic stage control 125 over a signal line 170, to store images captured by the CCD camera 135 in the memory 145, and to analyze images in accordance with the invention.

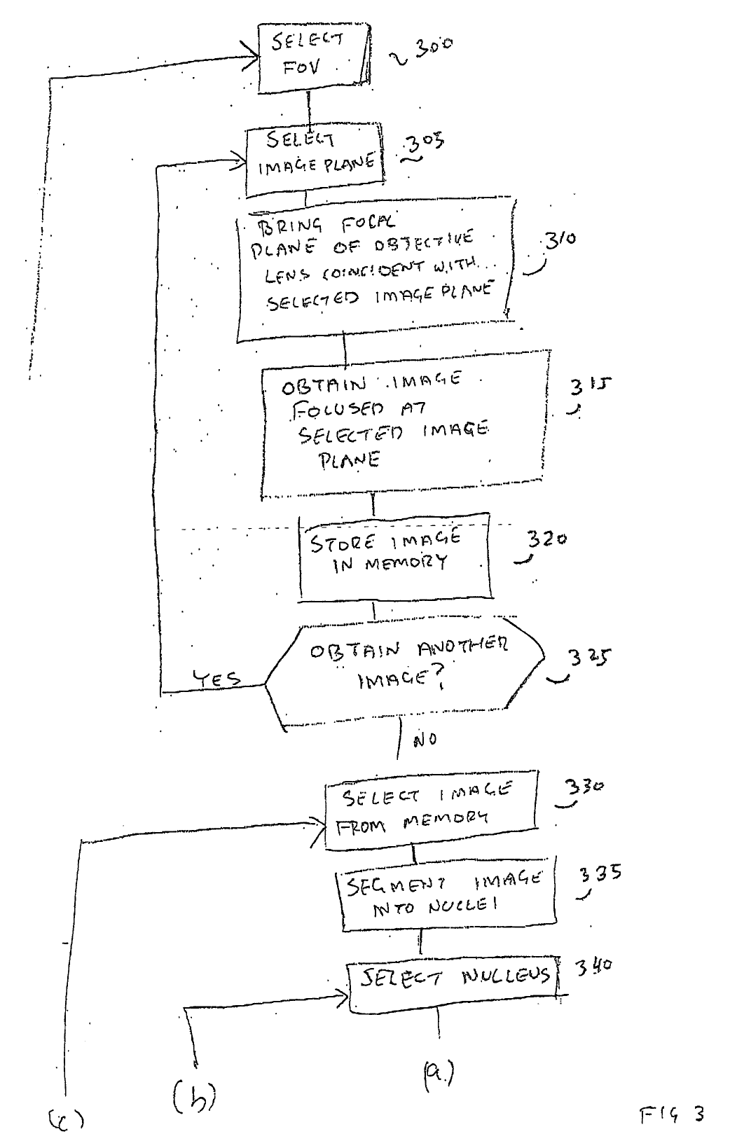

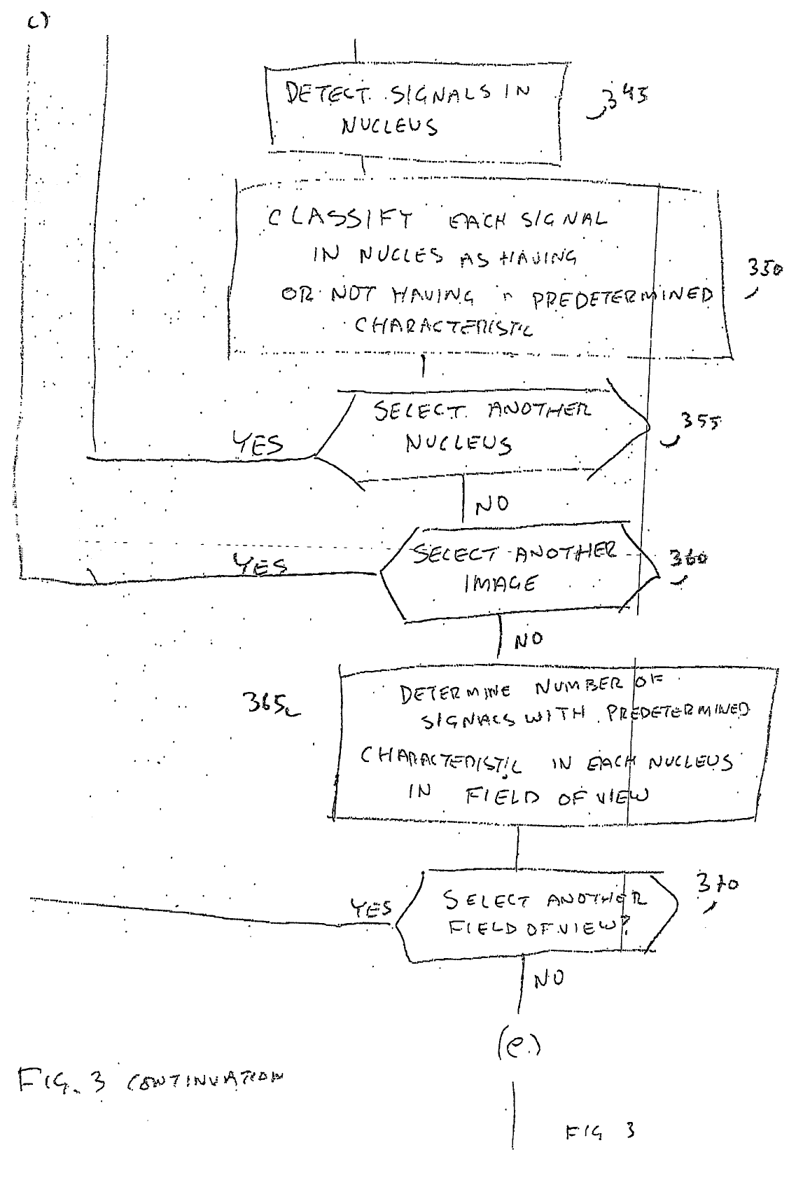

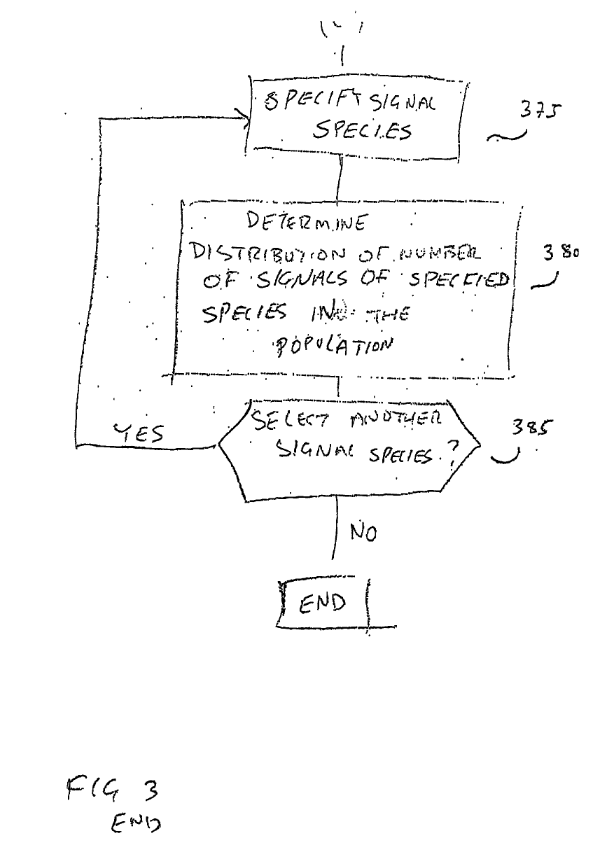

FIG. 3 shows a flow chart of an algorithm carried out by the processor 165 in accordance with one embodiment of he invention. In step 300 a field of view is selected in the specimen 110. In step 305, the location of a plane at which an image of the specimen 110 is to be obtained is determined. The processor 130 then activates the electronic stage control 125 in order to bring the determined plane coincident with the focal plane of the objective lens 120 (step 310). An image at the determined plane is obtained (step315) and the image is stored in the memory 145 (step 320). At step 325, it is determined whether another image being recalled from the plane in the same field of view is to be obtained. If yes, the process returns to step 305. If no, the process continues at step 330 with an image being recalled from the memory. The image is segmented into spaces (e.g. cells or nuclei) in step 335, and in step 340 a space in the field of view is selected. Then, in step 345, optical signals are detected in the selected space. Each detected signal is then classified as having or not having a predetermined property (step 350). The predetermined property may be for example that the signal is focused in the image. In this case, the detection step 345 and the classification step 350 may be performed using a signal detection and classification algorithms as disclosed in Leaner, et al., 2001. In step 355 it is determined whether another space in the field of view is to be selected. If yes, the process returns to step 340. If no, then in step 360 it is determined whether another image is to be selected from the memory. If yes, the process returns to step 330. If no, then in step 365 the number of signals having the predetermined characteristics in each selected space in the field of view is determined. Then, in step 370, it is determined whether another field of view is to be analyzed. If yes, the process returns to step 300. If no, the process continues at step 375 with the specification of a signal species to be analyzed. The signal may be specified, for example, by specifying its spectral properties. In step 380 the distribution of the number of signals of the specified type per space having the predetermined property is determined. In step 385, it is determined whether another signal species is to be analyzed. If yes, the process returns to step 375. If no, the process terminates.

It will also be understood that the system according to the invention may be a suitably programmed computer. Likewise, the invention contemplates a computer program being readable by a computer for executing the method of the invention. The invention further contemplates a machine-readable memory tangibly embodying a program of instructions executable by the machine for executing the method of the invention.

### EXAMPLE

The method of the invention was applied to FISH dot counting performed simultaneously for the detection of trisomies 21 and 13.

Specimen Preparation

The interphase nuclei preparations from amniotic fluid were made using the method of Klinger et al., 1996 with minor modifications. 1-2 ml of amniotic fluid was centrifuged and the cell pellet washed in PBS warmed to 37°. The cells were resuspended in 75 mM Potassium Chloride (KCl) and put directly on to slides coated with APES (Sigma) and incubated at 37° for 15 minutes. Evaporation of PBS was compensated with filtered distilled water. Excess fluid was carefully removed and replaced with 100 ml of 3% Carnoys fixative, 70% 75 mM KCl at room temperature for 5 minutes. The excess fluid was carefully removed and 5 drops of fresh fixative were dropped on to the cell area. Slides were briefly dried on a 60° hotplate, and then either used immediately for hybridization or dehydrated through an alcohol series and stored at −20° until required.

Hybridization

Target areas were marked on the slides using a diamond tipped scribe. Target DNA was denatured by immersing in 70% formamide:30% 2×SSC at 73° for 5 minutes. 10 μL of probe mix containing spectrum orange LSI 21 and spectrum green LSI 13 (Vysis, Downers Grove, Ill.) was applied to the target area and a coverslip placed over the probe solution. Coverslips were sealed using rubber cement and slides placed in a pre-warmed humidified container in a 37° incubator for 16 hours. Coverslips were removed and slides washed in 0.4×SSc/0.3% NP-40 solution at 73° for 2 minutes. Slides were then placed in 2×SSC/0.1% NP-40 solution at room temperature for 1 minute. When completely dried 10 μL of DAPI II counterstain (Vysis, Downers Grove, Ill.) was applied to the target area and sealed under a coverslip.

Instrumentation and Screening Procedure

Slides were screened under a Zeiss axioplan epifluorescence microscope using Zeiss ×100 objective. Signals were viewed using Vysis DAPI/Green/Orange triple bandpass filter set and images acquired using a CCD camera (Photometrics CH250/A) and SmartCapture software (Vysis, Downers Grove, Ill.). Red and green signals, corresponding to chromosomes 21 and 13 respectively, were seen on blue DAPI stained nuclei. Images were collected from five slides and stored in TIFF (Tagged Image File Format) format.

Multi-Spectral Image Analysis

Multiple probes, labelled by different fluorophores, were used in conjunction, i.e., chromosomes 13 and 21 are indicated by green and red signals, respectively, while the nuclei were stained blue. Colour was kept and specified by the RGB (red, green, blue) format, where each image pixel is represented by the normalised red, green and blue brightness values. DAPI nuclei are analysed in the blue channel of the RGB image, whereas red (spectrum orange; chromosome 21) and green (spectrum green; chromosome 13) signals are analysed separately in the red and green channels, respectively. Multi-spectral image analysis facilitates pre-processing and segmentation, and yields hue-based features, which are efficient for FISH signal representation and classification. It also allows the analysis of multiple probes.

Colour image segmentation is performed separately on each of the three different channels of the RGB image using global thresholds with values of 0.5 and 0.8 of the maximum channel intensity for the segmentation of signals and nuclei, respectively. Following thresholding, noise reduction, boundary smoothing of the nuclei by morphological operations and spatio-spectral correlation between nuclei and signals are implemented to complete the segmentation of the nuclei and signals.

Signal Feature Measurement

The features measured for each of the segmented signals were area (a size measure), eccentricity (a shape measure), total and average channel intensities (intensity measures), intensity standard deviation (texture measure), as well as color measures derived following the conversion of the RGB format into the HSI (hue, saturation, intensity) format.

Signal Classification

Focused and unfocused signals of two probes (spectrum orange and spectrum green) are represented here using the above features and classified into four classes—‘focused red’, ‘unfocused red’, ‘focused green’ and ‘unfocused green’. The classifier in this example is based on a two-layer perception neural network as disclosed in Bishop, 1995.

Dot Counting

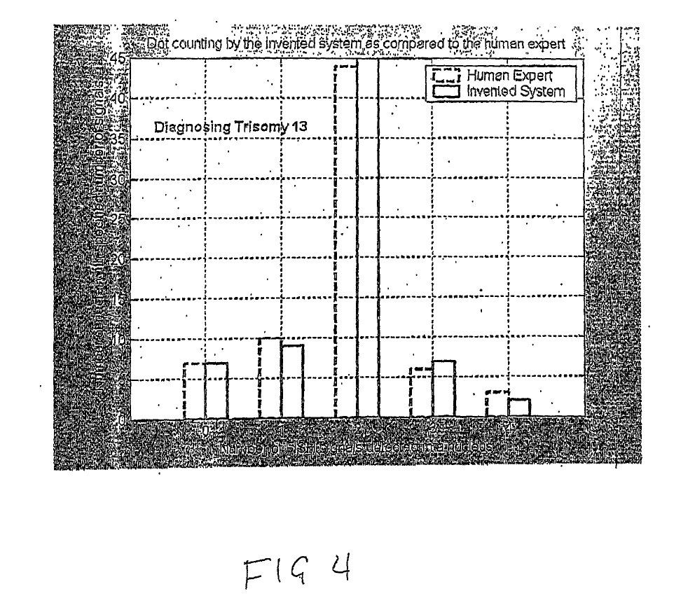

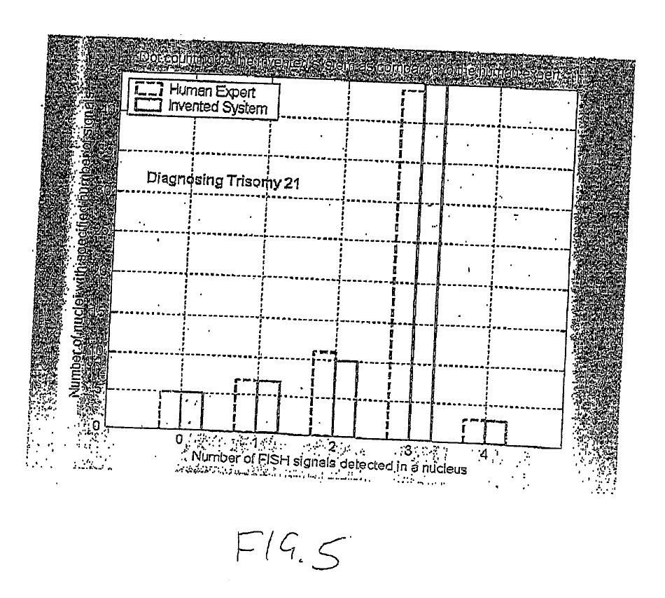

Images of 70 nuclei were captured and analysed by the system as described above and used to diagnose trisomy 13 and 21 in a subject with trisomy 21. The proportion of cells having a specific number of signals was determined. Dot counting results obtained in accordance with the invention were compared with those of a human expert obtained by visual inspection of the specimens, which was considered as a golden standard. The results are shown in FIG. 4 with respect to trisomy 13 and in FIG. 5 with respect to trisomy 21. Only small differences were observed between the values obtained in accordance with the invention and those obtained by the human expert. (5.7% in FIG. 4 and 2.8% in FIG. 5). Both the method of the invention and the expert correctly diagnosed the normality with respect to trisomy 13 and abnormality with respect to trisomy 21.

1. A method for analyzing one or more three dimensional spaces, comprising, for each three dimensional space:
(a) for each of the one or more DNA sequences,
(aa) labeling copies of the DNA sequence in the space with a probe producing a detectable optical signal;
(ab) for each of a plurality of parallel planes passing through the space,
(aba) obtaining a two-dimensional image of the space that is focused in the plane; and
(abb) determining the number of optical signals from the DNA sequence in the image having one or more predetermining characteristics; and
(ac) determining the total number of optical signals in the images having the one or more predetermined characteristics.

2. The method according to claim 1 wherein the three dimensional space is a cell, nucleus or virus.

3. The method according to claim 1 wherein a probe is a fluorescent probe.

4. The method according to claim 1 wherein the predetermining one or more characteristics includes the signal being in focus in the image.

5. The method according to claim 1 wherein determining the number of optical signals in an image having one or more predetermined characteristics is performed using a multi-class classifier of signal patterns.

6. The method according to claim 5 wherein the signal classifier is based on a two-layer perceptron neural network.

7. The method according to claim 1 further comprising determining whether a DNA sequence has begun replication based upon a distribution of the total number of optical signals in the three-dimensional spaces having the one or more predetermined characteristics.

8. The method according to claim 1 further comprising determining whether a DNA sequence has completed replication based upon a distribution of the total number of optical signals in the three-dimensional spaces having the one or more predetermined characteristics.

9. The method according to claim 1 further comprising determining whether a three-dimensional space exhibits aneuploidy for a DNA sequence.

10. The method according to claim 8 wherein the aneuploidy is trisomy.

11. The method according to claim 9 wherein the DNA sequence is a locus on any one of human chromosomes 13, 18, 21, X and Y.

12. The method according to claim 9 wherein the aneuoploidy is indicative of a disorder selected from the group comprising:
(a) a neoplasia, hematological malignancy, or a solid tumor; and
(b) male decreased fertility.

13. The method according to claim 1 further comprising determining whether a space exhibits sub-chromosomal deletion or duplication for a DNA sequence.

14. The method according to claim 13 wherein the deletion or duplication is indicative of a disorder selected from the group comprising:
(a) mental retardation or congenital malformation;
(b) follow up of offspring of translocation carriers; and
(c) neoplasia.

15. The method according to claim 1 further comprising determining whether a three-dimensional space exhibits gene amplification of a DNA sequence.

16. The method according to claim 15 wherein gene amplification is indicative of a cancer or Charcot-Marie-Tooth disease.

17. The method according to claim 1 further comprising determining whether a three-dimensional space exhibits micro-deletion for a DNA sequence.

18. The method according to claim 17 wherein the micro-deletion is indicative of DiGeorge or Miller-Deker syndrome.

19. The method according to claim 1 further comprising determining whether a three-dimensional space exhibits an irregularity of signal conformation during replication of a DNA sequence.

20. The method according to claim 19 wherein the irregularity in replication is indicative of loss of a mono-allelic imprinted mode of gene replication selected from the group comprising:
(a) Angelman/prader-Willi syndrome; and
(b) Renal cell carcinoma.

21. The method according to claim 19 wherein the irregularity in replication is indicative of loss of a Mendelian expected bi-allelic imprinted mode of gene replication selected from the group comprising:
(a) leukemia or lymphoma;
(b) renal cell carcinoma;
(c) carcinoma of the cervix; and
(d) autosomal trisomies.

22. The method according to claim 19 wherein irregularity in replication is indicative of tri-nucleotide amplification of fragile-X mental retardation syndrome (FMR1).

23. A system for analyzing one or more three-dimensional spaces, comprising:
(a) an optical system configured, for each of a pluraity of parallel planes passing through a three-dimensional space, to obtain a two-dimensional image of the three dimensional space that is focused in the plane; and
(b) a processor configured to:
(ba) for each of a plurality of two-dimensional images of a three-dimensional space, to determine the number of optical signals from a DNA sequence in the image having one or more predetermined characteristics when the DNA sequence is labeled with a probe producing the optical signal;
(bb) to determine the total number of optical signals from the DNA sequence in the images having the one or more predetermined characteristics.

24. The system according to claim 23 wherein the three dimensional space is a cell, nucleus or virus.

25. The system according to claim 23 wherein a probe is a fluorescent probe.

26. The system according to claim 23 wherein the predetermined one or more characteristics includes the signal being in focus in the image.

27. The system according to claim 23 wherein determining the number of optical signals in an image having one or more predetermined characteristics is performed using a multi-class classifier of signal patterns.

28. The system according to claim 27 wherein the signal classifier is based on a two-layer perceptron neural network.

29. The system according to claim 23 wherein the processor is further configured to determine whether a DNA sequence has begun replication based upon a distribution of the total number of optical signals in the three-dimensional spaces having the one or more predetermined characteristics.

30. The system according to claim 23 wherein the processor is further configured to determine whether a DNA sequence has completed replication based upon a distribution of the total number of optical signals in the three-dimensional spaces having the one or more predetermined characteristics.

31. The system according to claim 23 wherein the processor is further configured to determine whether a three-dimensional space exhibits aneuploidy for a DNA sequence.

32. The system according to claim 31 wherein the aneuploidy is trisomy.

33. The system according to claim 31 wherein the DNA sequence is a locus on any one of human chromosomes 13, 18, 21, X and Y.

34. The system according to claim 9 wherein the aneuploidy is indicative of a disorder selected from the group comprising:
(c) a neoplasia, hematological malignancy, or a solid tumor; and
(d) male decreased fertility.

35. The system according to claim 23 wherein the processor is further configured to determine whether a space exhibits sub-chromosomal deletion or duplication for a DNA sequence.

36. The system according to claim 35 wherein the deletion or duplication is indicative of a disorder selected from the group comprising:
(a) mental retardation or congenital malformation;
(d) follow up of offspring of translocation carriers; and
(e) neoplasia.

37. The system according to claim 23 wherein the processor is further configured to determine whether a three-dimensional space exhibits gene amplification of a DNA sequence.

38. The system according to claim 37 wherein gene amplification is indicative of a cancer or Charcot-Marie-Tooth disease.

39. The system according to claim 23 wherein the processor is further configured to determine whether a three-dimensional space exhibits micro-deletion for a DNA sequence.

40. The system according to claim 39 wherein the micro-deletion is indicative of DiGeorge or Miller-Deker syndrome.

41. The system according to claim 23 wherein the processor is further configured to determine whether a three-dimensional space exhibits an irregularity of signal conformation during replication of a DNA sequence.

42. The system according to claim 41 wherein the irregularity in replication is indicative of loss of a mono-allelic imprinted mode of gene replication selected from the group comprising:
(c) Angelman/prader-Willi syndrome; and
(d) Renal cell carcinoma.

43. The system according to claim 41 wherein the irregularity in replication is indicative of loss of a Mendelian expected bi-allelic imprinted mode of gene replication selected from the group comprising:
(e) leukemia or lymphoma;
(f) renal cell carcinoma;
(g) carcinoma of the cervix; and
(h) autosomal trisomies.

44. The system according to claim 41 wherein irregularity in replication is indicative of tri-nucleotide amplification of fragile-X mental retardation syndrome (FMR1).

45. A method for optimizing a classifier, the classifier being configured, for each of N quantifiable features, to determine a value of the feature associated with an object, the method comprising:
(a) Determining a number n of features, n being n?N, that minimizes a classifier probability function of miscalculation; and
(b) selecting n features from among the N features that maximize a ratio of between-class scatter to the within-class scatter.

46. A method for analyzing one or more three dimensional spaces, comprising, for each three dimensional space:
(a) for each of the one or more DNA sequences,
(aa) labeling copies of the DNA sequence in the space with a probe producing a detectable optical signal;
(ab) for each of a plurality of parallel planes passing through the space,
(aba) obtaining a two-dimensional image of the space that is focused in the plane; and
(abb) determining the number of optical signals from the DNA sequence in the image having one or more predetermined characteristics; and
(ac) determining the total number of optical signals in the images having the one or more predetermined characteristic.

47. A system for analyzing one or more three-dimensional spaces, comprising:
(a) an optical system configured, for each of a plurality of parallel planes passing through a three-dimensional space, to obtain a two-dimensional image of the three dimensional space that is focused in the plane; and
(b) a processor configured to:
(ba) for each of a plurality of two-dimensional images of a three-dimensional space, to determine the number of optical signals from a DNA sequence in the image having one or more predetermined characteristics when the DNA sequence is labeled with a probe producing the optical signal;
(bb) to determine the total number of optical signals from the DNA sequence in the images having the one or more predetermined characteristics.

48. A method for optimizing a classifier, the classifier being configured, for each of N quantifiable features, to determine a value of the feature associated with an object, the method comprising:
(a) Determining a number n of features, n being n?N, that minimizes a classifier probability function of miscalculation; and
(b) selecting n features from among the N features that maximize a ratio of between-class scatter to the within-class scatter.
